(19) United States
(12) Patent Application Publication
(10) Pub. No.: US 2005/0159368 A1
(43) Pub. Date: Jul. 21, 2005

(54) Wound treatment composition and process of manufacture

(76) Inventor: Stanley Johnston, Los Angeles, CA (US)

Correspondence Address: Anderson & Morishita, L.L.C., Suite 102, 2725 S. Jones Blvd., Las Vegas, NV, 89146, US

(21) Appl. No.: 10/761,837

(22) Filed: Jan. 20, 2004

(51) Int. Cl. A61K 31/7048; A61K 31/47; A61K 31/555; A61K 31/573; A61K 31/137

(52) U.S. Cl. 514/28; 514/185; 514/311; 514/649; 514/171

(57) ABSTRACT

A wound treatment composition contains one or more antimicrobial agents that alone or in combination have antibacterial and antifungal properties, a vasoconstriction agent, a steroidal anti-inflammatory, a stimulant, and a carrier. The wound treatment composition may optionally include a demulcent agent and a skin disinfectant agent. A process of manufacturing a wound treatment composition is also disclosed.

### FIELD OF THE INVENTION

This invention relates to wound treatment compositions and processes of manufacturing wound treatment compositions. More specifically, the present invention is a wound treatment composition which, when applied to wounds, shortens healing time. The present invention is also a process of manufacturing such a wound treatment composition.

### BACKGROUND OF THE INVENTION

Everybody suffers injuries, trauma, and wounds at some point in his or her lifetime. “Wounds” could refer to those that break the skin, such as punctures, incisions, lacerations, abrasions, and the like, as well as those that damage the skin without necessarily breaking the skin such as blistering, contusions, burns, and the like. As can be appreciated, wounds can result from many different types of trauma and wear to the skin. Treatment of wounds could range from no treatment for a minor cut or scrape, to treatment with disinfectant for a more serious cut, to physical suturing for the most serious wounds.

The body's normal response to a wound has three stages: inflammatory; fibroblastic; and maturation. During inflammation, components for healing gather at the wound site. Therefore, prior art references state that inflammation is desirable. In fact, the prior art states that anti-inflammatories are not useful in healing wounds and actually tend to slow the healing process. Next, fibroblasts positioned at the wound site begin generating collagen, the physical structure that will close the wound. During the maturation phase, collagen replacement continues and the wound may begin to fade as scarring becomes less pronounced.

During the healing process, the patient often experiences pain, itching, tenderness, swelling, scarring, or other discomfort. Wound healing time can depend upon many factors including the severity of the wound. This process can range from a few days to several weeks or months. However, infection can extend this time or, in the most severe cases, cause necrosis of nearby tissue or even death.

Generally, wound treatments are intended to protect the wound from infection and, thereby, promote healing. In other words, most wound treatments are actually directed to suppression or prevention of infection and wound healing is merely a byproduct of the antibiotic wound treatment. There are few, if any, wound treatments that shorten the healing time through components directed to wound healing.

However, healing time can be key for certain professions. For example, professional athletes must perform at a high level on a regular basis. Extended healing time can cause the athlete to miss playing time. Moreover, some athletes, like baseball pitchers, are prone to chronic wear on the skin that can cause blistering. This can hamper the athlete's performance on a continuous and chronic basis.

### SUMMARY OF THE INVENTION

A composition according to the present invention includes one or more antimicrobial agents that alone or in combination have antibacterial and antifingal properties. In an optional embodiment, the antimicrobial agents include one or more of Polymyxin B Sulfate, Bacitracin Zinc, and 8-Hydroxyquinoline Sulfate. For example, the composition could include substantially 25 percent by weight of a mixture of Polymyxin B Sulfate and Bacitracin Zinc in a ratio of 20 parts Polymyxin B Sulfate to 1 part Bacitracin Zinc and 0.3 percent by weight of 8-Hydroxyquinoline Sulfate.

The composition further comprises a vasoconstriction agent. Optionally, the vasoconstriction agent is Phenylephrine HCL. In one example embodiment, the composition includes 0.25 percent by weight Phenylephrine HCL.

Also, the composition includes a steroidal anti-inflammatory. Optionally the steroidal anti-inflammatory is micronized Hydrocortisone. For example, in one optional embodiment, the composition includes substantially 1 percent by weight micronized Hydrocortisone.

Additionally, the composition includes a stimulant. In an optional embodiment, the stimulant is Ichthammol. Thus, in one optional embodiment, the composition includes substantially 6.25 percent by weight Ichthammol.

The composition is delivered by a carrier. Optionally the carrier is a base ointment. The base ointment may take many different forms but in an optional embodiment, the base ointment includes anhydrous lanolin and white petrolatum. In a further optional embodiment, one part anhydrous lanolin ointment is combined with three parts white petrolatum.

The composition of the present invention may optionally include skin disinfectant agents and demulcent agents. For example, a skin disinfectant agent such as Povidone-Iodine may be included. Similarly, a demulcent agent such as tincture of Benzoin could be included. In an example embodiment, the composition includes 12.8 percent by weight Povidone-Iodine and 11.7 percent tincture of Benzoin.

A summary of one optional embodiment is given in Table 1:

TABLE 1

| | Percentage |
|---|---|
| Component | by Weight |
| Base Ointment | 42.70 |
| Polymyxin B Sulfate and Bacitracin Zinc in a ratio of 20 | 25.00 |
| parts Polymyxin B Sulfate to 1 part Bacitracin Zinc | |
| 8-Hydroxyquinoline Sulfate 0.3% | 0.30 |
| Benzoin Tincture | 11.70 |
| Ichthammol | 6.25 |
| Hydrocortisone (micronized) | 1.00 |
| Phenylephrine HCL 0.25% | 0.25 |
| Povidone-Iodine 10% Solution | 12.80 |

The present invention also includes a process for manufacturing a composition and a product produced by that process. The process includes mixing a base ointment, where the base ointment includes substantially one part anhydrous lanolin to three parts white petrolatum. Liquid components Povidone-Iodine (substantially 12.8% by weight), tincture of Benzoin (substantially 11.7% by weight), and Ichthammol (substantially 6.25% by weigh) are combined with base ointment and mixed. The remaining agents are combined, optionally by titurating the powder components. The remaining components include substantially 25% by weight Polymyxin B Sulfate and Bacitracin Zinc in a ratio of 20 parts Polymyxin B Sulfate to 1 part Bacitracin Zinc, substantially 0.3% by weight 8-Hydroxyquinoline Sulfate 0.3%, substantially 1% by weight Hydrocortisone (micronized), and substantially 0.25% by weight Phenylephrine HCL 0.25%. This mixture of components is added to mixture of base ointment and liquid components.

### BRIEF DESCRIPTION OF THE DRAWINGS

FIG. 1 shows a flow chart of a method of manufacture according to an embodiment of the present invention.

### DESCRIPTION

The wound treatment composition of the present invention includes one or more antimicrobial agents that alone or in combination have antibacterial and antifungal properties, a vasoconstriction agent, a steroidal anti-inflammatory, a stimulant, and a carrier. An optional embodiment of the invention may also include a skin disinfectant agent and/or a demulcent agent.

Any antimicrobial agent or combination of antimicrobial agents may be used. However, it is contemplated that the antimicrobial agent or agents have antibacterial and antifungal properties. For example, the antimicrobial agents may include one or more of Bacitracin Zinc, Polymyxin B Sulfate, 8-Hydroxyquinoline Sulfate, or other antimicrobial agents. In one optional embodiment, for example, the wound treatment composition may include Bacitracin Zinc, Polymyxin B Sulfate, and 8-Hydroxyquinoline Sulfate. While these antimicrobial agents could be included in any effective quantity, an optional embodiment includes substantially 25 percent by weight of a mixture of Polymyxin B Sulfate and Bacitracin Zinc in a ratio of 20 parts Polymyxin B Sulfate to 1 part Bacitracin Zinc. Such an optional embodiment of a wound treatment composition may also include substantially 0.3 percent by weight of 8-Hydroxyquinoline Sulfate 0.3%.

An agent causing vasoconstriction is also included. Such a vasoconstriction agent is not included for the ends of vasoconstriction but as a means of slowing absorption of other co-administered agents. Any vasoconstriction agent may be used. In an optional embodiment, Phenylephrine HCL is included. In one optional embodiment, the composition includes substantially 0.25 percent by weight Phenylephrine HCL 0.25%.

A steroidal anti-inflammatory is also included in the wound treatment composition. Optionally, a steroidal anti-inflammatory is selected to stabilize lysosomal membranes to prevent the release of lytic enzymes that could extend tissue damage during inflammation and generate leukotactic substances. The steroidal anti-inflammatory may take any form, but in an optional embodiment, micronized Hydrocortisone is used. Such an embodiment could optionally include substantially 1 percent by weight Hydrocortisone. It is noted that the present invention may reveal a synergy of Hydrocortisone and Polymyxin B Sulfate that aids in suppressing secondary bacterial invasion.

A wound treatment composition according to the present invention also includes a stimulant. Optionally, any stimulant could be used to promote absorption of swellings and effusions. However, in an optional embodiment, Ichthammol is used. While not necessary, it is noted that Ichthammol is also an antibacterial agent. In an optional embodiment including Ichthammol, the wound treatment composition may include substantially 6.25 percent by weight Ichthammol.

The active agents of the wound treatment composition are delivered by a carrier. While the carrier could take any form, including liquid, creme, gel, suspension, gas, or solid, optionally, the carrier is an ointment. In an optional embodiment, the ointment includes anhydrous lanolin ointment and white petrolatum. It is noted that anhydrous lanolin also has an emollient property and its low water content allows the liquid agents to be incorporated. Also, anhydrous lanolin provides increased absorption of the active agents. However any base ointment may be used that serves to maintain a uniform consistency and holds the active ingredients in contact with the affected area.

In addition to these agents, a wound treatment composition according to the present invention may also include one or more of a skin disinfectant agent or agents and a demulcent agent or agents. For example, in an optional embodiment, a wound treatment composition may include a skin disinfectant such as Povidone-Iodine. In one optional embodiment including Povidone-Iodine, substantially 12.8 percent by weight Povidone-Iodine 10% solution. Additionally or alternatively, a wound treatment composition may optionally include a demulcent agent such as tincture of Benzoin. In an optional embodiment, a wound treatment composition includes substantially 11.7 percent by weight tincture of Benzoin.

Thus, one optional embodiment of the present invention may take the form summarized in Table 2:

TABLE 2

| | Percentage |
|---|---|
| Component | by Weight |
| Base Ointment | 42.70 |
| Polymyxin B Sulfate and Bacitracin Zinc in a ratio of 20 | 25.00 |
| parts Polymyxin B Sulfate to 1 part Bacitracin Zinc | |
| 8-Hydroxyquinoline Sulfate 0.3% | 0.30 |
| Benzoin Tincture | 11.70 |
| Ichthammol | 6.25 |
| Hydrocortisone (micronized) | 1.00 |
| Phenylephrine HCL 0.25% | 0.25 |
| Povidone-Iodine 10% Solution | 12.80 |

Of course, such ratios are merely illustrative of one optional embodiment and should not be considered limiting. More specifically, it is noted that the agents may be included in any amount so long as the agent is effective to accomplish the function described in the claims. Also, it is noted that the present invention contemplated that additional agents may be added to the list of claimed agents.

The present invention also includes a process for manufacturing a wound treatment composition. Such a process may include preparing the carrier. In the case of a wound treatment composition that includes a base ointment, the base ointment is prepared 10. As noted above, in one optional embodiment, the base ointment may comprise anhydrous lanolin and white petrolatum. To prepare such an optional form of the base ointment, the anhydrous lanolin ointment and white petrolatum are mixed until smooth and uniform.

Liquid components are incorporated 12 into the base ointment. These may include the Povidone-Iodine, tincture of Benzoin, and Ichthammol, if these components are used. In such an optional embodiment, substantially 12.8% by weight Povidone-Iodine 10% solution, substantially 11.7% by weight tincture of Benzoin in liquid form, and substantially 6.25% by weight Ichthammol in liquid form are combined with said base ointment. The combination is mixed until smooth and the liquid has been incorporated.

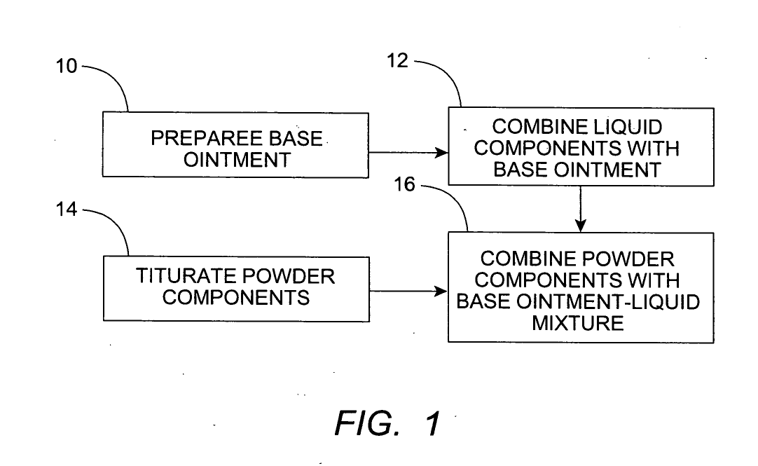

The remaining components are combined. In an optional embodiment in which agents are in powder form, the powdered components are fiturated 14 and added 16 to the base ointment-liquid mixture. In an optional embodiment, substantially 25% by weight Polymyxin B Sulfate and Bacitracin Zinc in a ratio of 20 parts Polymyxin B Sulfate to 1 part Bacitracin Zinc, substantially 0.3% by weight 8-Hydroxyquinoline Sulfate 0.3%, substantially 1% by weight Hydrocortisone (micronized), and substantially 0.25% by weight Phenylephrine HCL 0.25% are combined. This combination is then added to the mixture of base ointment and Povidone-Iodine, tincture of Benzoin, and Ichthammol. This process is summarized in FIG. 1. While certain embodiments of the present invention have been shown and described it is to be understood that the present invention is subject to many modifications and changes without departing from the spirit and scope of the claims presented herein.

1. A composition comprising:
one or more antimicrobial agents that alone or in combination have antibacterial and antifungal properties;
a vasoconstriction agent;
a steroidal anti-inflammatory;
a stimulant; and
a carrier.

2. The composition of claim 1 wherein said antimicrobial agents comprise one or more of the agents selected from the group of Polymyxin B Sulfate, Bacitracin Zinc, and 8-Hydroxyquinoline Sulfate.

3. The composition of claim 2 wherein said antimicrobial agents comprise substantially 25.3 percent by weight of the composition.

4. The composition of claim 1 wherein said vasoconstriction agent is Phenylephrine HCL.

5. The composition of claim 4 wherein said vasoconstriction agent comprises substantially 0.25 percent by weight of said composition.

6. The composition of claim 1 wherein said steroidal anti-inflammatory is micronized Hydrocortisone.

7. The composition of claim 6 wherein steroidal anti-inflammatory comprises substantially 1 percent by weight of said composition.

8. The composition of claim 1 wherein said stimulant is Ichthammol.

9. The composition of claim 8 wherein said stimulant comprises substantially 6.25 percent by weight of said composition.

10. The composition of claim 1 further comprising a skin disinfectant agent.

11. The composition of claim 10 wherein said skin disinfectant agent is Povidone-Iodine.

12. The composition of claim 11 wherein said skin disinfectant agent comprises substantially 12.8 percent by weight of said composition.

13. The composition of claim 1 further comprising a demulcent agent.

14. The composition of claim 13 wherein said demulcent agent is tincture of Benzoin.

15. The composition of claim 14 wherein said demulcent agent comprises substantially 11.7 percent by weight of said composition.

16. The composition of claim 1 wherein said carrier is a base ointment.

17. The composition of claim 16 wherein said base ointment is a mixture including anhydrous lanolin and white petrolatum.

18. A composition comprising the formulation by weight:

| | Percentage |
|---|---|
| Component | by Weight |
| Base Ointment | 42.70 |
| Polymyxin B Sulfate and Bacitracin Zinc in a ratio of 20 | 25.00 |
| parts Polymyxin B Sulfate to 1 part Bacitracin Zinc | |
| 8-Hydroxyquinoline Sulfate 0.3% | 0.30 |
| Benzoin Tincture | 11.70 |
| Ichthammol | 6.25 |
| Hydrocortisone (micronized) | 1.00 |
| Phenylephrine HCL 0.25% | 0.25 |
| Povidone-Iodine 10% solution | 12.80 |

19. The composition of claim 18 wherein said base ointment is a mixture including anhydrous lanolin and white petrolatum.

20. A process for manufacturing a composition comprising:
mixing a base ointment, said base ointment comprising substantially one part anhydrous lanolin to three parts white petrolatum;
combining substantially 12.8% by weight Povidone-Iodine 10% solution, substantially 11.7% by weight tincture of Benzoin in liquid form, and substantially 6.25% by weight Ichthammol in liquid form with said base ointment and mixing; and
combining substantially 25% by weight Polymyxin B Sulfate and Bacitracin Zinc in a ratio of 20 parts Polymyxin B Sulfate to 1 part Bacitracin Zinc, substantially 0.3% by weight 8-Hydroxyquinoline Sulfate 0.3%, substantially 1% by weight Hydrocortisone (micronized), and substantially 0.25% by weight Phenylephrine HCL 0.25% and adding to said mixture of base ointment and Povidone-Iodine, tincture of Benzoin, and ichthammol.

21. A product made according to the process of claim 20.
